(19) United States
(12) Patent Application Publication
(10) Pub. No.: US 2014/0199344 A1
(43) Pub. Date: Jul. 17, 2014

(54) ANTI-MEASLES CANCER IMMUNOTHERAPY

(71) Applicant: MOR RESEARCH APPLICATIONS LTD., Tel-Aviv (IL)

(72) Inventors: Shmuel ARIAD, Lehavim (IL); Jacob GOPAS, Beer Sheva (IL); Daniel BENHARROCH, Ramat Hanegev (IL); Irina LAZAREV, Beer Sheva (IL)

(73) Assignee: MOR RESEARCH APPLICATIONS LTD., Tel-Aviv (IL)

(21) Appl. No.: 14/217,741

(22) Filed: Mar. 18, 2014

Related U.S. Application Data
(63) Continuation of application No. 12/741,447, filed on Sep. 13, 2010.
(60) Provisional application No. 60/985,293, filed on Nov. 5, 2007.

(51) Int. Cl. A61K 39/165; A61N 5/10

(52) U.S. Cl. CPC A61K 39/165; A61N 5/10
USPC 424/212.1

(57) ABSTRACT

The present invention provides methods for treating specific populations of cancer patients by immunotherapy. The methods comprise administering an immunogenic composition eliciting an immune response to measles virus to patients having measles virus-immunoreactive cancer cells.

### FIELD OF THE INVENTION

The present invention relates to the field of cancer immunotherapy. More specifically, the present invention relates to methods of treating cancer by enhancing an immune response to measles virus in cancer patients having measles immunoreactivity in their cancer cells.

### BACKGROUND OF THE INVENTION

Measles virus (MV) is an enveloped negative strand RNA virus classified in the family Paramyxoviridae in the genus Morbillivirus whose genome encodes six protein products, the N (nucleocapsid), P (polymerase cofactor phosphoprotein), M (matrix), F (fusion), H (hemagglutinin) and L (large RNA polymerase) proteins. The H protein is a surface glycoprotein which mediates measles virus attachment to its receptor, CD46 (Dorig, et al., Cell 1993, 75: 295-305). The F protein is responsible for cell-cell fusion after viral attachment has taken place. Measles virus has a natural tropism for lymphoid cells and, in particular, cancerous lymphoid cells. Measles virus may cause persistent viral infection and a number of persistently infected cell lines have been investigated. It has been suggested that persistent infection is possible only by mutated, and not by the wild type MV (Rima, B K and Duprex, P W. Virus Research, 2005, 111:132-147).

MV is the cause of subacute sclerosing panencephalitis (SSPE), a human CNS disease that manifests itself long after the acute infection with the virus. MV infection is associated with neurological complications in a small minority of cases. About 1:1000 cases will suffer post-infection encephalitis, which involves mainly perivascular demyelination (Litvak et al., Am. J. Dis. Child, 1943, 65:265-95). This often fatal complication appears to be autoimmune in nature as no virus can be demonstrated in the brain of such patients. SSPE has been reported to occur in 1:300,000 cases. However, more recent data suggests that it is much more prevalent and can follow acute MV infection in 1:10,000 cases (Takasu et al., Epidemiol. Infect., 2003, 131:887-98). On average, symptoms present 8 years after the acute infection, but this ranges from 9 months to 30 years. The disease manifests itself in severe demyelination and profound infection of neurons. In the latter stages of SSPE small numbers of oligodendrocytes, astrocytes and endothelial cells have been shown to be affected (Kirk et al., Neuropathol. Appl. Neurobiol., 1991, 17:289-97). This inevitably leads to severe neurological deficits and death of the patient. One of the longest recorded patients carried the persistent infection over three decades before the onset of symptoms. There is no evidence for a reduction in the cell-mediated immune responses to MV in SSPE patients. Furthermore, antibody responses lead to hyperimmunity with extremely high titers of neutralizing antibody in both the serum and cerebrospinal fluid (CSF) of patients. The CSF contains oligoclonal bands specific to MV and it has been demonstrated that the antibodies present in these have undergone affinity maturation (Smith-Jensen et al., Neurology, 2000, 54:1227-1232).

In immunocompromised patients, measles can give rise to an additional CNS infection, measles inclusion body encephalitis (MIBE) (ter Meulen et al., In: Fraenkel-Conrat, H., Wagner, R. R. (Eds.), Comprehensive Virology, vol. 18. Plenum Press, New York, 1983, pp. 105-59.). In contrast to SSPE, MIBE is not associated with a hyperimmune antibody response to measles proteins or oligoclonal bands in the CNS. Other suggested sequelae of MV infection are multiple sclerosis, chronically active autoimmune hepatitis, Paget's disease, otosclerosis, Crohn's disease and autism among many other diseases (e.g. Reddy et al., Exp. Hematol, 1999, 27:1528-32).

Oncolytic viruses are being developed as anticancer therapy. They propagate selectively in tumor tissue and destroy it without causing excessive damage to normal non-cancerous tissues (Russel and Peng, Trends Pharmacol. Sci., 2007, 28:326-33). Numerous viruses are now being considered as potential cancer therapeutics, including the attenuated strains of measles virus used in vaccines (Fielding, Rev. Med. Virol., 2005, 15:135-42; Nakamura and Russel, Expert Opin. Biol. Ther., 2004, 4:1685-92; Iankov et al., Mol. Ther., 2007, 15:114-22; Blechacz et al., Hepatology, 2006, 44:1465-77; McDonald et al., Breast Cancer Res. Treat., 2006, 99:177-84; Myers et al., Cancer Gene Ther., 2005, 12:593-9).

A method for limiting the growth of cancer cells by administering an attenuated measles virus directly into a tumor or systemically by injection is disclosed in U.S. Pat. No. 7,117,740. This method uses the oncolytic properties of MV to provide the anti-cancer therapeutic effect. This disclosures neither teaches nor suggests utilizing the immunogenic properties of MV or compositions comprising MV antigens to induce or augment an immune response to cancer cells with the aim of providing an anti-cancer immunotherapeutic effect.

A few human viruses have been detected in human cancers and established as oncogenic. Human papilloma virus (HPV) is associated with cervical cancer, Epstein-Barr virus (EBV) with B-cell lymphoproliferative diseases, KSHV with Kaposi's sarcoma and primary effusion lymphoma, HTLV-1 with T-cell leukemia, and Hepatitis C Virus and Hepatitis B Virus with hepatocellular carcinoma (for a complete review see, Pagano et al., Semin Cancer Biol., 2004, 14:453-71).

There is indirect evidence indicating that MV may be associated with cancer. MV-phosphoprotein inhibits ubiquitination of Pirh2, one of the factors responsible for degrading the cell cycle regulator p53 (Chen et al., J. Virol., 2005, 79:11824-36). In another study, Pirh2 was overexpressed in lung cancer biopsies compared with normal lung tissue (Duan et al., J. Nat. Cancer Inst., 2004, 96:1718-21). In addition, the MV receptor CD46 is overexpressed in LC cells (Varsano S, et al., Clin. Exp. Immunol., 1998, 113:173-82) and has been detected in human breast cancer biopsies (Thorsteinsson et al., AMPIS, 1998; 106:869-78).

Direct evidence for MV implication in Hodgkin's lymphoma (HL) is conflicting. A study of 154 patients showed positive immunostaining with at least two anti-measles antibodies in biopsies of more than half of the patients (Benharroch et al., Br. J. Cancer, 2004, 91:572-9). However, two later studies found no evidence for MV antigens or MV genome or transcripts (Wilson et al., Int. J. Cancer, 2007, 121:442-7.; Maggio et al., Int. J. Cancer, 2007, 121:448-53).

Current cancer therapies include surgery, radiation, chemotherapy, and immunotherapy. Immunotherapy attempts to harness the power of the hosts' immune system and direct it to attack the cancerous cells. Some forms of immunotherapy use vaccines composed of antigens derived from tumor cells to boost the body's production of antibodies or T lymphocytes. Non-specific immunotherapy has been successful in a limited number of cases (e.g. BCG for the treatment of urinary bladder cancer, IL-2 for the treatment of melanoma and renal cancer). Passive immunotherapy in the form of antibodies, and particularly monoclonal antibodies, has been the subject of considerable research and development as anti-cancer agents. Several monoclonal antibodies for tumor therapy have been registered as pharmaceuticals for human use, e.g. Erbitux (ImClone/Bristol-Myers Squibb), Panorex (Centocor/Glaxo-Wellcome), Avastin (Genentech/Roche), Rituxan (Biogen IDEC/Genentech/Roche) and Herceptin (Genentech/Roche).

Specific active immunotherapy targets antigens that are exclusively or preferentially associated with cancer cells, namely tumor specific antigens (TSA) or tumor associated antigens (TAA), and to use such antigens or fractions thereof as the basis for an immune attack on the tumor. Another approach aims to cure or contain the disease with cancer vaccines, by training the patient's immune system to recognize the cancer cells by presenting it with highly antigenic and immunostimulatory cellular debris. Initially cancer cells are harvested from the patient (autologous cells) or from established cancer cell lines (allogeneic) and then are grown in vitro. These cells are then engineered to be more recognizable to the immune system by the addition of one or more genes, which are often cytokine genes that produce pro-inflammatory immune stimulating molecules, or highly antigenic protein genes. These altered cells are grown in vitro and killed, and the cellular contents are incorporated into a vaccine. Immunotherapy is also being attempted through the delivery of immunostimulatory genes, mainly cytokines, to the tumor in vivo.

Despite recent advances in cancer therapy, new therapy options need to be developed. Five-year survival rates for pancreatic cancer (4%), lung cancer (15%), liver cancer (7%) and glioblastoma (5%) remain low (Cancer Facts and Figures 2006. American Cancer Society Web site).

There is an unmet medical need for more efficient cancer therapies with improved safety profiles, particularly for cancer therapies tailored to specific types of molecules and specific patients populations.

### SUMMARY OF THE INVENTION

The present invention provides methods of treating cancer by eliciting an immune response to measles virus (MV) in cancer patients having detectable measles virus in at least part of their cancer cells. Typically the detection is performed by immunoassay or by detecting MV derived nucleic acids. According to the principles of the present invention immunogenic compositions capable of eliciting an immune response to measles virus are useful to bolster the patient's immune response to the cancer cells.

It is herein disclosed that MV is detectable in cancer cells present in an unexpectedly large proportion of cancer patients. This surprising finding is observed in several types of cancers, including breast carcinoma, small-cell lung carcinoma, non-small cell lung carcinoma, thymic carcinoma, and endometrial cancer. According to the present invention measles virus or antigenic components thereof are disclosed as relevant target antigens for cancer immunotherapy in this MV-positive patient sub-population. The present invention accordingly provides a method for treating cancer in patients that have MV-positive cancer cells by enhancing anti-measles immunity.

The present invention is therefore based in part on the detection of MV in the cancerous cells of a subject and on treatment directed towards enhancing the patient's immune response against measles as immunotherapy for cancer. The present invention now discloses the use of immunogenic compositions capable of eliciting an immune response to MV in cancer. In particular the present invention discloses immunotherapy for patients having MV-positive cancer cells. MV may be detected in cancer cells present in a solid tumor or in a biological fluid or a biopsy.

The advantages of the present invention are numerous, including the fact that most cancer patients undergo a biopsy of the tumor tissue, and therefore the tissue sample needed to determine MV immunoreactivity in the cancerous cells is readily available. Furthermore, the necessary immunohistochemistry tests are readily performed by clinical laboratories. Finally, one of the major advantages of the immunotherapeutical treatment of the present invention is the availability of commercial measles vaccines, together with the long history of safety of said vaccines in human use.

According to one aspect, the present invention provides a method of treating cancer in patients comprising the steps of: (a) obtaining a sample of cancer cells from said patients, (b) testing said sample for immunoreactivity to measles virus, (c) detecting the measles virus-immunoreactive cancers, and (d) administering an immunogenic composition eliciting an immune response against a measles virus to the patients having the measles virus-immunoreactive cancer cells detected in step (c).

In some embodiments the MV-positive cells are detected by immunohistochemistry. Any antibody that specifically recognizes an MV antigen may be used to detect the MV-positive cancers. Anti-MV nucleoprotein and anti-MV hemagglutinin are currently preferred. In other embodiments the MV-positive cells are detected by nucleic acid-based detection methods such as mRNA amplification or in situ hybridization.

According to this aspect of the present invention, the patients having cancerous cells harboring MV are treated by administering an immunogenic composition eliciting an immune response against a measles virus. The active ingredient of the immunogenic composition may include any component able to initiate, sustain, or boost an immune response of the patient to MV. In some embodiments, the immunogenic composition comprises RNA-free virus-like particles. In other embodiments the immunogenic composition comprises at least one isolated MV protein or portion thereof, selected from the group consisting of the six proteins encoded by the MV genome, namely, the nucleoprotein (NP), the polymerase cofactor phosphoprotein (P), the matrix (M), the fusion (F), the hemagglutinin (H), and the large RNA polymerase (L). According to certain embodiments the antigenic protein is delivered by means of a DNA vaccine.

In some embodiments, the immunogenic composition further comprises an immunostimulatory substance. In some embodiments, the immunostimulatory substance is a Toll-like receptor (TLR) inducer. In certain embodiments the TLR inducer is selected from the group consisting of: a CpG-containing oligonucleotide, an immunostimulatory nucleic acid, a lipopolysaccharide, a peptidoglycan, a lipoteichoic acid, an imidazoquinoline compound, a flagellin, a lipoprotein, and an immunostimulatory organic molecule. In a currently preferred embodiment, the TLR inducer is a CpG-containing oligonucleotide. In some embodiments, the immunogenic composition is administered together with an adjuvant.

In other embodiments of the present invention, the immune response is elicited by administration of a composition comprising a complete measles virus particle. The virus may be selected from the Edmonston Zagreb measles strain, the Edmonston-Enders strain, the Moraten strain, and the Moraten Berna strain. In some embodiments, the immunogenic composition comprises a killed measles virus. In other embodiments, the immunogenic composition comprises an attenuated measles virus. According to some embodiments, the immunogenic composition is provided as a commercial vaccine formulation. According to specific embodiments, the commercial vaccine may comprise additional viruses such as the MMR-II vaccine, which comprises an attenuated measles virus, an attenuated mumps virus, and an attenuated rubella virus.

In some embodiments the immunogenic composition comprises an edible vaccine. In edible vaccines, the antigen eliciting the immune response is expressed in transgenic plant material such as a fruit or vegetable. The plant material containing the antigen is processed in such manner as to be suitable for human consumption. According to specific embodiments, the transgenic plant is selected from the group consisting of tobacco, rice, lettuce, bananas, and figs.

In some embodiments the immunogenic composition is administered only once to the patient. In alternative embodiments, at least one booster treatment is further administered. In some embodiments the immunogenic composition is administered in a single dose. In other embodiments, it is administered in repeated doses.

In several embodiments the method of treating cancer further comprises treating the subject with other anti-cancer therapies. Such additional therapies include, inter alia, chemotherapy, radiation therapy, surgery, hormone therapy, other immunomodulatory therapies, therapy with targeted or antiangiogenic agents, or other therapies approved for cancer treatment. According to various embodiments the immune composition is administered before, concurrently with, or after the additional therapy.

In currently preferred embodiments, a booster vaccination is administered after each cycle of chemotherapy. In other currently preferred embodiments, a booster vaccination is administered concurrently with each cycle of chemotherapy or another immunomodulatory, targeted or antiangiogenic agents or radiation therapy.

According to various embodiments, the immunogenic composition is administered to the patient intramuscularly, subcutaneously, transdermally, orally, or intranasally. In certain embodiments the immunogenic composition is administered intramuscularly, subcutaneously, or transdermally.

Typically, the cancer is characterized in that at least a portion of the cancer cells express one or more measles virus epitopes, or at lease one epitope that is immunologically cross-reactive with a measles virus epitope. For example, without limitation, the cancer may be brain cancer, prostate cancer, breast cancer, skin cancer, colon cancer, rectal cancer, lung cancer, pancreatic cancer, Hodgkin's lymphoma, non-Hodgkin's lymphoma, multiple myeloma, leukemia, oesophageal cancer, renal cancer, uterine, endometrial and cervical cancer, ovarian cancer, testicular cancer, urinary bladder cancer, gastric cancer, liver cancer, thyroid cancer, and head and neck cancer. In various particular embodiments, the cancer may be brain cancer, prostate cancer, breast cancer, skin cancer, colon cancer, rectal cancer, lung cancer, pancreatic cancer, oesophageal cancer, renal cancer, uterine, endometrial and cervical cancer, ovarian cancer, testicular cancer, urinary bladder cancer, gastric cancer, liver cancer, thyroid cancer, and head and neck cancer, wherein each possibility represents a separate embodiment of the present invention. In other particular embodiments, the cancer is breast carcinoma, small-cell lung carcinoma, non-small cell lung carcinoma, thymic carcinoma, or endometrial cancer, wherein each possibility represents a separate embodiment of the present invention.

In currently preferred embodiments the method is used to treat breast cancer, lung cancer, colorectal cancer, endometrial cancer, and Hodgkin lymphoma. In other currently preferred embodiments, the cancer is breast cancer or non-small cell lung carcinoma.

According to another aspect, the present invention provides a method of treating cancer in patients comprising administering an immunogenic composition capable of eliciting an immune response against a measles virus, wherein the immunogenic composition does not comprise viable measles virus. Within this aspect of the invention, the immunogenic composition may be selected from the group consisting of: RNA-free virus-like particles, at least one isolated MV protein or portion thereof selected from the group consisting of the six proteins encoded by the MV genome, a measles virus DNA vaccine, a killed measles virus, or an edible vaccine.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 shows tissue sections of breast cancer biopsies stained with antibodies to MV antigens (IHC with diaminobenzidine, ×240 magnification) (A) Invasive breast adenocarcinoma case negative for the L77 anti-hemagglutinin antibody. (B) Invasive breast adenocarcinoma case mildly positive (+1) for the L77 anti-hemagglutinin antibody. (C) Invasive breast adenocarcinoma case strongly positive (+2) for the L77 anti-hemagglutinin antibody. (D) Intraductal breast carcinoma case positive for the L77 anti-hemagglutinin antibody. (E) Invasive breast adenocarcinoma case negative for the NP 39/22 anti-nucleoprotein antibody. (F) Invasive breast adenocarcinoma case mildly positive (+1) for the NP 39/22 anti-nucleoprotein antibody. (G) Invasive breast adenocarcinoma case strongly positive (+2) for the NP 39/22 anti-nucleoprotein antibody. (H) Intraductal breast carcinoma case positive for the NP 39/22 anti-nucleoprotein antibody.

FIG. 2 shows tissue sections of lung cancer biopsies stained with antibodies to MV antigens (IHC with diaminobenzidine, ×240 magnification). (A1) Lung squamous cell carcinoma case negative for the NP 39/22 anti-nucleoprotein antibody. (A2) Lung squamous cell carcinoma case mildly positive (+1) for the NP 39/22 anti-nucleoprotein antibody. (A3) Lung squamous cell carcinoma case strongly positive (+2) for the NP 39/22 anti-nucleoprotein antibody. (B1) Lung adenocarcinoma case negative for the L77 anti-hemagglutinin antibody. (B2) Lung adenocarcinoma case mildly positive (+1) for the L77 anti-hemagglutinin antibody. (B3) Lung adenocarcinoma case strongly positive (+2) for the L77 anti-hemagglutinin antibody.

### DETAILED DESCRIPTION OF THE INVENTION

The present invention is directed to the use of MV to induce an anticancer immunotherapeutic response. The present invention discloses an association of MV with breast cancer and non small cell lung cancer. It was previously reported that MV may be associated with Hodgkin's lymphoma (Benharroch et al., 2004)

The present invention now discloses the use of immunogenic components of MV in cancer immunotherapy for patients having MV-positive cells in a solid tumor or in a biological fluid. As defined herein, the terms “MV-immunoreactive cells”, “MV-immunoreactive cancer”, “MV-positive cells”, and “MV-positive cancer” are used interchangeably to denote a group of cancerous cells, or a tumor in which measles virus or components thereof have been detected, typically by immunological or nucleic acid-based methods, or any other method capable of detecting a measles virus infection.

As defined herein, the term “solid tumor” is a group of cancer cells which grows at an anatomical site outside of the blood stream and requires the formation of small blood vessels and capillaries to supply nutrients to the growing tumor mass.

As defined herein, the term “biological fluid” refers to any extracellular bodily fluid, including but not limited to blood, urine, saliva, interstitial fluid, lymph, and cerebrospinal fluid.

According to some embodiments of the invention, a sample of tissue containing cancerous cells is obtained from a patient by a surgical procedure, generally by excision, resection, or fine needle aspiration. The cancerous tissue sample is then tested for MV, which may be detected by nucleic acid-based assays such as mRNA amplification or in situ hybridization, or preferably, by immunohistochemistry. These methods are well known to the skilled artisan and practiced in clinical laboratories at medical centers.

The presence of measles virus epitopes may be determined in a cell or tissue sample obtained from the tumor, or, in alternate embodiments, in cell-containing specimens of body fluids, rinse fluids that were in contact with the primary tumor site, or tissues or organs other than the tissue primary tumor site (e.g. for testing tumor metastases).

For example, the presence of measles RNA may be detected by methods known in the art such as PCR, RT-PCR, in situ PCR, in situ RT-PCR, and hybridization with a probe comprising a detectable moiety. Such methodologies are well known to those skilled in the art and can be conveniently found in published laboratory methods manuals (e.g., Sambrook, J. et al., eds., MOLECULAR CLONING, A LABORATORY MANUAL, 2nd. edition, Cold Spring Harbor Laboratory Press, Cold Spring Harbor, N.Y. (1989); Ausubel, F. et al., eds., CURRENT PROTOCOLS 1N MOLECULAR BIOLOGY, John H. Wiley & Sons, Inc. (1997)).

In one embodiment MV mRNA is detected in the tissue sample by reverse-transcriptase polymerase chain reaction (RT-PCR). This method uses PCR amplification of relatively rare RNA molecules. First, RNA molecules are purified from cells and converted into complementary DNA (cDNA) using a reverse transcriptase enzyme (such as an MMLV-RT) and primers such as oligo-dT, random hexamers, or gene-specific primers. Then by applying gene-specific primers and Taq DNA polymerase, a PCR amplification reaction is carried out in a PCR machine. Those of ordinary skill in the art are capable of selecting the length and sequence of the gene-specific primers and the PCR conditions (i.e., annealing temperatures, number of cycles, and the like) that are suitable for detecting specific RNA molecules. It will be appreciated that a semi-quantitative RT-PCR reaction can be employed, by adjusting the number of PCR cycles and comparing the amplification product to known controls.

In another embodiment, MV mRNA is detected by in situ hybridization. In this method DNA or RNA probes are attached to the RNA molecules present in the cells. Generally, the cells are first fixed to microscopic slides to preserve the cellular structure and to prevent the RNA molecules from being degraded, and then are subjected to hybridization buffer containing the labeled probe. The hybridization buffer includes reagents such as formamide and salts (e.g., sodium chloride and sodium citrate) which enable specific hybridization of the DNA or RNA probes with their target mRNA molecules in situ while avoiding non-specific binding of probe. Those skilled in the art are capable of adjusting hybridization conditions (i.e., temperature, concentration of salts and formamide, and the like) for specific probes and types of cells. Following hybridization, any unbound probe is washed off and the slide is subjected to either a photographic emulsion, which reveals signals generated using radio-labeled probes, or to a colorimetric reaction, which reveals signals generated using enzyme-linked labeled probes.

In other embodiments, the presence of a measles virus epitope is detected, for example, by an immunoassay. Various methods for detecting the presence of proteins and antigenic fragments thereof are known in the art, and include, for example, Enzyme-linked immunosorbent assay (ELISA), Western blot, fluorescence-activated cell sorting (FACS), immunohistochemical analysis or using antigen array-based immunoassays. Such techniques are well known to the ordinarily skilled artisan and have been described in many standard immunology manuals and texts, e.g., Harlow, E. and Lane, D., “Antibodies: A Laboratory Manual,” Cold Spring Harbor Laboratory, Cold Spring Harbor, N.Y. (1988); Deutscher, M. P., “Guide to Protein Purification,” Meth. Enzymol. 128, Academic Press San Diego (1990); Scopes, R. K., “Protein Purification Principles and Practice,” 3rd ed., Springer-Verlag, N.Y. (1994). Typically, an immunoassay involves the step of determining the capacity of an antibody to specifically bind a MV antigen, which may be performed, for example, by quantifying specific antigen-antibody complex formation. The term “specifically bind” as used herein means that the binding of an antibody to an antigen is not competitively inhibited by the presence of non-related molecules.

### Enzyme-Linked Immunosorbent Assay (ELISA)

This method involves fixation of a sample containing a protein substrate (e.g., fixed cells or a proteinaceous solution) to a surface such as a well of a microtiter plate. A substrate-specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

### Western Blot

This method involves separation of a substrate from other protein by means of an acrylamide gel followed by transfer of the substrate to a membrane (e.g., nitrocellulose, nylon, or PVDF). Presence of the substrate is then detected by antibodies specific to the substrate, which are in turn detected by antibody-binding reagents. Antibody-binding reagents may be, for example, protein A or secondary antibodies. Antibody-binding reagents may be radiolabeled or enzyme-linked, as described hereinabove. Detection may be by autoradiography, colorimetric reaction, or chemiluminescence. This method allows both quantitation of an amount of substrate and determination of its identity by a relative position on the membrane indicative of the protein's migration distance in the acrylamide gel during electrophoresis, resulting from the size and other characteristics of the protein.

### Radioimmunoassay (RIA)

In one version, this method involves precipitation of the desired protein (i.e., the substrate) with a specific antibody and radiolabeled antibody-binding protein (e.g., protein A labeled with I125) immobilized on a precipitable carrier such as agarose beads. The radio-signal detected in the precipitated pellet is proportional to the amount of substrate bound.

In an alternate version of RIA, a labeled substrate and an unlabelled antibody-binding protein are employed. A sample containing an unknown amount of substrate is added in varying amounts. The number of radio counts from the labeled substrate-bound precipitated pellet is proportional to the amount of substrate in the added sample.

Fluorescence-Activated Cell Sorting (FACS)

This method involves detection of a substrate in situ in cells bound by substrate-specific, fluorescently labeled antibodies. The substrate-specific antibodies are linked to fluorophores. Detection is by means of a cell-sorting machine, which reads the wavelength of light emitted from each cell as it passes through a light beam. This method may employ two or more antibodies simultaneously.

Immunohistochemical Analysis

In a currently preferred embodiment, MV infection is detected in the cancer cells by immunohistochemistry (IHC). This method involves detection of a substrate in situ in fixed cells by substrate-specific antibodies. The substrate specific antibodies may be enzyme-linked or linked to fluorophores. Detection is by microscopy, and is either subjective or by automatic evaluation. With enzyme-linked antibodies, a colorimetric reaction may be required. It will be appreciated that immunohistochemistry is often followed by counterstaining of the cell nuclei, using, for example, hematoxyline or Giemsa stain.

Any antibody able to specifically recognize an MV antigen in an immunohistochemical analysis may be used in the methods of the present invention in order to detect MV-immunoreactive cancers. In currently preferred embodiments antibodies to the MV nucleoprotein or to MV hemagglutinin are used for this purpose.

In another embodiment, there is provided a method for treating cancer in a patient in need thereof; comprising a) determining whether the patient is afflicted with a measles virus-positive cancer, and b) administering an immunogenic composition eliciting an immune response to measles virus to said patient. In various embodiments, step a) may be perfected, for example, using an immunoassay or a nucleic acid based assay, wherein presence of at least one MV epitope or of MV RNA in a cancer cell-containing specimen obtained from the patient is indicative that the patient is afflicted with a measles virus-positive cancer. In one embodiment, a cancer cell-containing specimen obtained from the patient specifically binds an antibody directed to measles-virus (i.e. an antibody that specifically binds a MV epitope). For example, the cell-containing specimen may be immunoreactive with a measles virus nucleoprotein (NP) antigen or a measles virus hemagglutinin (HA) antigen.

In one embodiment a sample of cancer cells is defined as being MV immunoreactive if at least 10% of the cells in the sample are detected by the antibody. In another embodiment at least 20% of the cells in the sample are detected by the antibody. In another embodiment at least 30% of the cells in the sample are detected by the antibody. In yet another embodiment at least 50% of the cells in the sample are detected by the antibody. It will be appreciated by one skilled in the art that confidence in the results of the test will be attained by the use of several antibodies to different antigens and of appropriate controls.

The method according to the invention comprises administering a therapeutically effective amount of an immunogenic composition eliciting an immune reaction against MV in a patient having an MV-immunoreactive cancer. The term “immunogenicity” or “immunogenic” relates to the ability of a substance to stimulate or elicit an immune response. Thus, within the context of the present invention, an immunogenic composition is a pharmaceutical composition that comprises at least one substance capable of eliciting or stimulating an immune response. Immunogenicity is measured, for example, by determining the presence of antibodies specific for the substance. The presence of antibodies is detected by methods known in the art, for example using an ELISA assay.

In one embodiment the immunogenic composition comprises RNA-free MV virus-like particles. Virus-like particles (VLP) are obtained by transfection of avian cells with cDNA encoding major structural viral proteins in such a way as to produce self-assembling particles resembling infectious virions. However, these particles do not contain viral mRNA and are therefore not infectious. In this embodiment the MV-VLP are produced according to the method disclosed in US Pat. Appl. 2007/0178120, which teaches a method to produce VLP from paramyxoviruses, including measles virus, that are effective vaccines.

In a further embodiment, the immunogenic composition comprises at least one isolated MV protein, selected from the group consisting of the nucleoprotein (NP), the polymerase cofactor phosphoprotein (P), the matrix (M), the fusion (F), the hemagglutinin (H), and the large RNA polymerase (L) proteins. The proteins may be isolated by purification from a large-scale virus culture, may be produced by recombinant methods, or produced by chemical synthesis.

In one embodiment, the immunogenic composition comprises a DNA vaccine. DNA-based vaccines use bacterial plasmids to express protein immunogens in vaccinated hosts. Recombinant DNA technology is used to clone cDNAs encoding immunogens of interest into eukaryotic expression plasmids. Vaccine plasmids are then amplified in bacteria, purified, and directly inoculated into the hosts being vaccinated. DNA typically is inoculated by a needle injection of DNA in saline, or by a gene gun device that delivers DNA-coated gold beads into skin. The plasmid DNA is taken up by host cells, the vaccine protein is expressed, processed and presented in the context of self-major histocompatibility (MHC) class I and class II molecules, and an immune response against the DNA-encoded immunogen is generated. Two approaches to DNA delivery are injection of DNA in saline using a hypodermic needle or gene gun delivery of DNA-coated gold beads. Saline injections deliver DNA into extracellular spaces, whereas gene gun deliveries bombard DNA directly into cells. The saline injections require much larger amounts of DNA (100-1000 times more) than the gene gun (Fynan et al., Proc. Natl. Acad. Sci. USA, 1993, 90:11478-82). These two types of delivery also differ in that saline injections bias responses towards type 1 T-cell help, whereas gene gun deliveries bias responses towards type 2 T-cell help (Feltquate et al., J. Immunol., 1997, 158:2278-84; Pertmer et al., J. Virol., 1996, 70:6119-25). DNAs injected in saline rapidly spread throughout the body. DNAs delivered by the gun are more localized at the target site. Following either method of inoculation, extracellular plasmid DNA has a short half life of about 10 minutes (Kawabata et al., Pharm. Res., 1995, 12:825-30; Lew et al., Hum. Gene Ther., 1995, 6:553). Vaccination by saline injections can be intramuscular (i.m.) or intradermal (i.d.). US Pat. Appl. 2007/0048861 discloses compositions and methods to generate an immune response to a variety of pathogens, including measles, using DNA-vaccines.

In one embodiment of the invention, the immunogenic composition comprises an attenuated strain of virus. As defined herein, the term “attenuated” means a virus which is immunologically related to the wild type measles virus (i.e., the virulent virus) but which is not itself pathogenic and does not produce a “classical measles disease,” and is not a wild type virus. An attenuated measles virus is replication-competent, in that it is capable of infecting and replicating in a host cell without additional viral functions supplied by, for example, a helper virus or a plasmid expression construct encoding such additional functions. As used herein, the terms “wild-type” or “wild-type virus” refer to the characteristics of a measles virus as it is found in nature which is pathogenic. As used herein, a “pathogenic measles virus” is one which produces classical measles disease. As defined herein, “classical measles disease” is a syndrome comprising fever, coryza, cough, conjunctivitis, followed by the appearance of a maculopapular rash (Koplik's spots) which occurs upon infection with a wild type measles virus in an individual who is not immune to the virus.

In this embodiment an attenuated MV is grown in culture to provide an immunogenic composition. Attenuated strains of viruses are obtained by serial passage of the virus in cell culture (e.g., in non-human cells), until a virus is identified which is immunogenic but not pathogenic. While wild type virus will cause fatal infection in marmosets, vaccine strains do not. In humans, infection with wild type viral strains is not generally fatal but is associated with classic measles disease. Classic measles disease includes a latent period of 10-14 days, followed by a syndrome of fever, coryza, cough, and conjunctivitis, followed by the appearance of a maculopapular rash and Koplik's spots (small, red, irregularly shaped spots with blue-white centers found inside the mouth). The onset of the rash coincides with the appearance of an immune response and the initiation of virus clearance. In contrast, individuals receiving an attenuated measles virus vaccine do not display classical measles symptoms. Attenuation is associated with decreased viral replication (as measured in vivo by inability to cause measles in monkeys), diminished viremia, and failure to induce cytopathological effects in tissues (e.g., cell-cell fusion, multinucleated cells). However, these biological changes have not been mapped to any single genetic change in the virus genome.

In one embodiment of the invention, the immunogenic composition comprises a killed measles virus. As defined herein, a “killed” virus retains its immunogenicity but has lost its capacity to replicate. Methods to kill a virus are well known in the art and generally consist in heat inactivation, or modification of the genetic material by chemical reaction or by irradiation. For example, a method for inactivating the MV by treatment with a composition comprising an ethyleneimine is disclosed in U.S. Pat. No. 5,891,705.

In some embodiments of the invention, an attenuated strain of measles virus which has been clinically tested as a vaccine for measles infection is used to provide an immunogenic composition. The Moraten attenuated form of the virus has been used world-wide as a vaccine and has an excellent safety record (Hilleman, et al., J. Am. Med. Assoc., 1968, 206:587-90,). Accordingly, in one embodiment of the invention, the Moraten strain is used to provide an immunogenic composition. The Moraten vaccine is commercially available from Merck® and is provided lyophilized in a vial which when reconstituted to 0.5 ml comprises 10³ pfu/ml. A vaccine against the Moraten Berna strain is available from the Swiss Serum Vaccine Institute, Berne.

In another embodiment of the invention, the Edmonston-B vaccine strain of measles virus is used (MV-Edm) (Enders and Peebles, Proc. Soc. Exp. Biol. Med., 1954, 86:277-86,). MV-Edm grows efficiently in tumor cells but its growth is severely restricted in primary cultures of human peripheral blood mononuclear cells, normal dermal fibroblasts, and vascular smooth muscle cells. A form of the Enders attenuated Edmonston strain is available commercially from Merck (Attenuvax®). Other attenuated measles virus strains are also encompassed within the scope of the invention, such as Leningrad-16, and Moscow-5 strains (Sinitsyna, et al., Res. Virol., 1990, 141:517-31), Schwarz strain (Fourrier, et al., Pediatrie, 1969, 24:97-8), 9301B strain (Takeda, et al. J. Virol., 1998, 72/11:8690-6), the AIK-C strain (Takehara, et al., Virus Res., 1992, 26:167 75), and those described in Schneider-Shaulies et al., PNAS, 1995, 92:3943-7, the entireties of which are incorporated by reference herein.

In another embodiment of the invention, the measles virus is provided within a vaccine formulation. In a specific embodiment, the mumps measles and rubella vaccine (MMR) is used. The MMR vaccine was introduced into the United States in 1972 and into the United Kingdom in 1998. Commercially available preparations of the MMR vaccine is obtainable from Merck, Pasteur Merieux Connaught, or SmithKline Beecham, and also contain the Moraten strain of attenuated measles virus at a minimum titer of 1 pfu/ml. It should be apparent to those of skill in the art that any clinically tested measles vaccine is acceptable for use in the invention, and is encompassed within the scope of the invention.

The immunogenic compositions of the present invention can be administered to a patient per se, or in a pharmaceutical composition where it is mixed with suitable carriers or excipients, and can further comprise an adjuvant. The term “adjuvant” as used herein refers to non-specific stimulators of the immune response or substances that allow generation of a depot in the host which when combined with the immunogenic composition of the present invention provide for an even more enhanced immune response. A variety of adjuvants can be used. Pharmaceutically acceptable adjuvants include, but are not limited to, alum, incomplete Freund's adjuvant, muramyl dipeptide, liposomes (e.g., Gelvac®), chitin microparticles, chitosan, cholera toxin subunit B, and lipid emulsions (e.g., Intralipid® and Lipofundin®). Alum is a preferred adjuvant for human use. The choice of the adjuvant will be determined in part by the mode of administration of the vaccine. As used herein, a “pharmaceutical composition” refers to a preparation of one or more of the active ingredients described herein with other chemical components such as physiologically suitable carriers and excipients.

An additional immunostimulatory effect may be obtained with many immunostimulatory substances, including Toll-like receptor (TLR) inducers, as these are known to enhance immuno stimulation of many compositions. Accordingly, in one embodiment of the invention, the immunogenic compositions of the present invention comprise at least one TLR inducer. Ten human toll-like receptors are known up to date. They are activated by a variety of ligands. TLR2 is activated by peptidoglycans, lipoproteins, lipopolysaccharides, lipoteichonic acid and Zymosan, and macrophage-activating lipopeptide MALP-2; TLR3 is activated by double-stranded RNA such as poly (LC); TLR4 is activated by lipopolysaccharide, lipoteichoic acids and taxol and heat-shock proteins such as heat shock protein HSP-60 and Gp96; TLR5 is activated by bacterial flagella, especially the flagellin protein; TLR6 is activated by peptidoglycans, TLR7 is activated by imiquimod and imidazoquinoline compounds, such as R-848, loxoribine and bropirimine and TLR9 is activated by bacterial DNA, in particular CpG-containing oligonucleotides. Ligands for TLR1, TLR8 and TLR10 are not known so far. In a currently preferred embodiment, the immunostimulatory effect is obtained with a CpG-containing oligonucleotide.

One example of such a molecule is CpG 7909 [PF-3512676] (also known as ProMune and Vaxlmmune), a synthetic 24-mer oligonucleotide containing 3 CpG motifs designed to specifically agonize the Toll-like receptor 9 (TLR9). It is being developed for the treatment of cancer as a monotherapy and in combination with chemotherapeutic agents, and it is also under development as an adjuvant for vaccines against cancer and infectious diseases. CpG 7909, acting through the TLR9 receptor present in B cells and plasmacytoid dendritic cells, stimulates human B-cell proliferation, enhances antigen-specific antibody production and induces interferon-alpha production, interleukin-10 secretion and natural killer cell activity.

The immunogenic composition of the present invention may also be administered in the form of an edible vaccine. In edible vaccines, the antigen eliciting the immune response is expressed in transgenic plant material Immunization is achieved after oral consumption of the plant material containing the antigen, which may be consumed unprocessed, e.g., in fresh fruit, vegetable, root, tuber, or leaf, or in a processed product such as juice, freeze-dried material, or dried fruit.

An edible vaccine for measles consisting in the MV hemagglutinin protein expressed in transgenic tobacco has been described in International Patent Application No. WO 01/52886. A method for producing edible vaccines for several bacterial and viral pathogens, including MV, in transgenic Dunaliella Salina algae, is described in US Patent Application 2003/0066107. International Patent Application No. WO 2005/115130 describes methods for producing genetically modified fig (Ficus) plants and their use for producing edible vaccines. Several methods for using transgenic plants for oral immunization have been disclosed, for example U.S. Pat. Nos. 5,484,719; 5,914,123; 5,612,487; 6,034,320; 6,136,320; 6,194,560; US Patent Applications 2003/0190332, 2005/0129704, US 2007/248596.

According to specific embodiments, the transgenic plant expressing the MV antigen used in the immunogenic composition of the present invention is selected from the group consisting of tobacco, rice, lettuce, bananas, and figs.

Immunogenic compositions of the present invention comprise a “sufficient amount” or a “therapeutically effective amount” of components mentioned herein, as needed. A “therapeutically effective amount” means that the administration of that amount to an individual, either in a single dose or as part of a series, is effective for treatment. This amount varies depending upon the health and physical condition of the individual to be treated, the capacity of the individual's immune system to synthesize antibodies, the size of the tumor, the formulation of the composition, the treating doctor's assessment of the medical situation, and other relevant factors. It is expected that the amount will fall in a relatively broad range that can be determined through routine trials. The effectiveness of treatment can be monitored by establishing an improved clinical income (e.g., a decrease in tumor size, more frequent remissions, complete or partial, or longer disease-free survival).

The treatment regime of a cancer patient comprises vaccinating the patient with the immunogenic composition on at least one occasion. In preferred embodiments at least one additional vaccination, herein referred to as a “booster vaccination” is given to the patient at a later time. Booster vaccinations are usually given in intervals between two and forty weeks. For a treatment regime comprising a booster vaccination, the immunogenic compound(s) included in the initial vaccination may or may not be similar or equivalent to the immunogenic compound(s) in the booster vaccination. For example, in some embodiments a commercial vaccine preparation such as the MMR-II vaccine is comprised in the initial and in the booster vaccinations. In alternative embodiments a commercial vaccine is used for the initial vaccination and an edible vaccine is used for booster vaccinations. In other embodiments an edible vaccine is used for initial and booster vaccinations. It will be appreciated that many combinations of the various embodiments of the immunogenic composition can be used for the vaccination regime of the patient.

The immunotherapeutic treatment according to the invention can be used in combination with other cancer therapies, such as surgery, chemotherapy, radiotherapy, hormone therapy, therapy with immunomodulatory agents, and therapy with targeted or antiangiogenic agents. In some embodiments the immunogenic composition is administered when cancer is first detected, before treatment with other therapies. In other embodiments the composition is administered concomitant with other therapies. In yet other embodiments, the composition is administered after treatment with other therapies. In currently preferred embodiments the immunogenic composition is administered after each cycle of chemotherapy or concurrently with another immunomodulatory, targeted or antiangiogenic agents or radiation therapy

The immunotherapy may, for example, be administered in a regime comprising:
(i) an initial vaccination occurring 2-4 weeks after surgery, or 2-4 weeks after chemotherapy or radiotherapy has commenced or has been completed, or concurrently with chemotherapy or radiotherapy.
(ii) booster vaccinations every subsequent third week, or 2-4 weeks after each cycle of chemotherapy.

When used in combination with chemotherapy, the anti-cancer agent can be a chemotherapeutic drug, such as, alkylators including, but not limited to, busulfan (Myleran, Busulfex), chlorambucil (Leukeran), ifosfamide (with or without MESNA), cyclophosphamide (Cytoxan, Neosar), glufosfamide, melphalan, L-PAM (Alkeran), dacarbazine (DTIC-Dome), and temozolamide (Temodar); anthracyclines, including, but not limited to doxorubicin (Adriamycin, Doxil, Rubex), mitoxantrone (Novantrone), idarubicin (Idamycin), valrubicin (Valstar), and epirubicin (Ellence); antibiotics, including, but not limited to, dactinomycin, actinomycin D (Cosmegen), bleomycin (Blenoxane), daunorubicin, and daunomycin (Cerubidine, DanuoXome); aromatase inhibitors, including, but not limited to anastrozole (Arimidex) and letroazole (Femara); bisphosphonates, including, but not limited to zoledronate (Zometa); cyclo-oxygenase inhibitors, including, but not limited to, celecoxib (Celebrex); estrogen receptor modulators including, but not limited to tamoxifen (Nolvadex) and fulvestrant (Faslodex); folate antagonists including, but not limited to methotrexate and tremetrexate; inorganic arsenates including, but not limited to arsenic trioxide (Trisenox); microtubule inhibitors (e.g. taxanes) including, but not limited to vincristine (Oncovin), vinblastine (Velban), paclitaxel (Taxol, Paxene), vinorelbine (Navelbine), epothilone B or D or a derivative of either, and discodermolide or its derivatives, nitrosoureas including, but not limited to procarbazine (Matulane), lomustine, CCNU (CeeBU), carmustine (BCNU, BiCNU, Gliadel Wafer), and estramustine (Emcyt); nucleoside analogs including, but not limited to mercaptopurine, 6-MP (Purinethol), fluorouracil, 5-FU (Adrucil), thioguanine, 6-TG (Thioguanine), hydroxyurea (Hydrea), cytarabine (Cytosar-U, DepoCyt), floxuridine (FUDR), fludarabine (Fludara), pentostatin (Nipent), cladribine (Leustatin, 2-CdA), gemcitabine (Gemzar), and capecitabine (Xeloda); osteoclast inhibitors including, but not limited to pamidronate (Aredia); platinum containing compounds including, but not limited to cisplatin (Platinol) and carboplatin (Paraplatin); retinoids including, but not limited to tretinoin, ATRA (Vesanoid), alitretinoin (Panretin), and bexarotene (Targretin); topoisomerase 1 inhibitors including, but not limited to topotecan (Hycamtin) and irinotecan (Camptostar); topoisomerase 2 inhibitors including, but not limited to etoposide, VP-16 (Vepesid), teniposide, VM-26 (Vumon), and etoposide phosphate (Etopophos); and tyrosine kinase inhibitors including, but not limited to imatinib (Gleevec).

Suitable routes of administration may, for example, include oral, rectal, transmucosal, especially transnasal, intestinal, or parenteral delivery, including intramuscular, and subcutaneous injections. In preferred embodiments, the methods of the present invention comprise amounts and routes of administration similar to those of commercial vaccines. For example, the MMR-II vaccine is typically injected in a 1000 TCID₅₀ (tissue culture infectious dose) dose subcutaneously. In some embodiments, the therapeutically effective amount is provided in repeated doses. Repeat dosing is appropriate in cases in which observations of clinical symptoms or tumor size or monitoring assays indicate either that a group of cancer cells or a tumor has stopped shrinking or that the immune response to MV is declining while the tumor is still present.

In some embodiments of the present invention a method of treating cancer in patients is provided, which comprises administering an immunogenic composition capable of eliciting an immune response against a measles virus, wherein the immunogenic composition does not comprise viable measles virus. Within this aspect of the invention, the immunogenic composition may be selected from the group consisting of: RNA-free virus-like particles, at least one isolated MV protein or portion thereof selected from the group consisting of the six proteins encoded by the MV genome, a measles virus DNA vaccine, a killed measles virus, or an edible vaccine.

The types of cancer that can be treated by the methods of the present invention include, but are not limited to, brain cancer, prostate cancer, breast cancer, skin cancer, colon cancer, rectal cancer, lung cancer, pancreatic cancer, Hodgkin lymphoma, Non-Hodgkin lymphoma, multiple myeloma, leukemia, oesophageal cancer, renal cancer, uterine endometrial and cervical cancer, ovarian cancer, testicular cancer, urinary bladder cancer, gastric cancer, liver cancer, head and neck cancer, and thyroid cancer. In preferred embodiments, the immunogenic compositions of the present invention are used to provide a treatment for breast cancer, lung cancer, colorectal cancer, endometrial cancer, and Hodgkin's lymphoma. In other currently preferred embodiments, the cancer is breast cancer or non-small cell lung carcinoma.

The present invention will now be illustrated by the following examples which are intended to be construed in a non-limitative fashion.

### EXAMPLES

### Example 1

### Evidence for Association of Measles Virus with Breast Cancer

A cross-sectional, retrospective, clinicopathological study on patients with breast cancer was performed in order to find evidence for a possible association of MV with breast cancer.

Patients and Methods

Study Population:

Newly diagnosed untreated female patients with early or locally-advanced breast cancer of the invasive-ductal adenocarcinoma type, diagnosed at the Soroka Medical Center during the years 1998-2005. The main group of patients consisted of 81 patients aged 50 years or younger. The control group consisted of 50 patients, aged above 50 years. This cut point for age was chosen as a surrogate marker for menopausal status. In each group, patients were selected in similar proportions according to two major characteristics: 1) estrogen receptor status, and 2) lymph node involvement.

Exclusion criteria were male gender, histological diagnosis of breast cancer other than invasive-ductal adenocarcinoma, bilateral breast cancer, or a previous history of another primary tumor. The list of patients was constructed from the computerized file of all breast cancer patients referred to the Department of Oncology during the study period. For each patient, data were extracted from her case file in the department of oncology, and the hospital computerized file. Only patients whose biopsies were of fair quality were included in the study. The study was conducted after the approval of the appropriate ethical review boards.

Data: Data included the following parameters:

1. Demographic information including age of the patient, ethnic origin, place of birth, birth-date, and for new corners, also year of immigration.

2. Information of primary clinical and pathological stage according to AJCC classification for breast cancer, 2002 version.

3. All the cases were reviewed and graded using Elston's modification of the Bloom-Richardson grading system (Elston C W and Ellis I O., Histopathology 1991; 19:403-10). For each case, a representative paraffin block containing tumor was chosen and sections were taken for immunohistochemical studies. In addition to invasive adenocarcinoma, all specimens were reviewed for a DCIS component. Standard immunohistochemical studies were performed for estrogen, progesterone, and HER2/neu receptors, ki67 proliferation index, and p53. For patients who received neo-adjuvant chemotherapy, pathological data included the state of response (pCR) after completing chemotherapy.

Immunohistochemistry: Immunohistochemical staining (IHC) by the avidin-biotin peroxidase complex method (Envision, DAKO) was used to assess MV-hemagglutinin, MV-nucleoprotein, estrogen, progesterone and HER2/neu receptors; p53 and ki67. The immunohistochemical procedure for MV antigens was as following: 4-μm tissue sections of paraffin-embedded tissue were mounted on glass slides coated with silane (Sigma Chemical Co, Saint Quentin, France). Sections were dried at temperature of 37° C. for 48 hours before deparaffinization. Deparaffinization was performed for 20 minutes and rehydrated with alcohol 100% and 90%, 3 minutes for each. After deparaffinization, slides were washed in distilled water, and incubated in 3% hydrogen peroxide in methanol for 15 min to block the endogenous peroxidase activity. After incubation slides were incubated in 10 mmol L-1 citrate buffer (pH 6) and heated in a microwave oven at 800 W for cycles of 15 min for antigenic exposure. After heating slides were removed from the oven, allowed to cool at room temperature for 30 minutes, and washed in PBS before staining. The primary antibody was applied to the slides and incubated at room temperature for 1 hour. After three washes in PBS, slides were treated with Envision anti-goat IgG biotin-labeled secondary antibodies (Envision; DAKO) at a dilution of 1:500 (diluted in 0.05% PBS-Tween 20) at room temperature for 1 hour. Bound antibodies were washed three times in PBS. Finally, Slides were stained with chromagen, 3,3 diaminobenzidine (Sigma Chemical), washed, counterstained with hematoxylin, dehydrated, treated with xylene, and mounted.

Antibodies: anti-measles: we used two anti-measles monoclonal antibodies, anti-nucleoprotein (L39/61), and anti-hemagglutinin (L77) for staining. Both anti-measles antibodies were available to us from Schneider-Schaulies et al. (Virology 1991; 182:703-11). The specificity of these antibodies was established on Western blot. A neuroblastoma cell line permanently infected by MV was used as a positive control and the same cell line, but without infection with MV, was used as a negative control. The L39/61 antibody was diluted in 1:100, and it showed typical cytoplasmic staining. The L77 antibody was diluted in 1:100 and similarly showed a cytoplasmic staining.

p53: To determine p53 expression we used monoclonal, mice, anti-human p53 antibody 1018, at dilution of 1:100 (Zymed, South San Francisco, Calif.). p53 was stained primarily in the nucleus.

Ki67: To determine proliferation index we used a monoclonal, mice antihuman MIBI antibody, at a dilution of 1:1000 (DAKO, Carpinteria, Ca).

Estrogen receptor (ER): To determine ER expression we used a monoclonal, mice antihuman 6F 11 antibody, at a dilution of 1:100 (Novocastra).

Progesterone receptor (PR): To determine PR expression we used a monoclonal, mice antihuman NCLPGR antibody, at a dilution of 1:100 (Novocastra).

HER2/neu: To determine HER2/neu expression we used a monoclonal, mice antihuman TAB250 antibody, at a dilution of 1:100 (Zymed, South San Francisco, Calif.).

Evaluation of staining: the intensity, staining percentage, and pattern of staining (nuclear and cytoplasmic) were assessed for the following markers: MV-hemagglutinin, MV-nucleoprotein, ER, PR, p53, ki67, and HER2/neu. The intensity was scored compared with background staining. The percentages of positive cells were estimated by calculating the ratio of the positively stained tumor cells to the total invasive tumor cells. Table 1 delineates the characteristics of markers considered positive on IHC.

TABLE 1
Consideration of positivity of the following markers:
ER, PR, p53, ki67, HER2/neu.

| Marker Type | Positive Staining Characteristics |
|---|---|
| ER | Strongly positive nuclear staining in >10% of tumor cells, |
| (estrogen | or weakly positive staining in >50% of tumor cells |
| receptor) | |
| PR | Strongly positive nuclear staining in >10% of tumor cells, |
| (proges- | or weakly positive staining in >50% of tumor cells |
| terone | |
| receptor) | |
| p53 | Strongly positive staining in >10% of tumor cells |
| Ki67 | Positive nuclear staining in >40% of tumor cells |
| Her2/neu | Weak to moderate complete membrane staining in >10% of |
| | tumor cells (+2), or strong complete membrane staining |
| | in >10% of tumor cells (+3) |

Evaluation of staining for MV antigens: The cytoplasmic intensity of MV-hemagglutinin and MV-nucleoprotein were evaluated separately and scored semi-quantitatively as negative (0, no expression or weakly-positive staining in less than or equal to 30% of the cells, or strongly-positive in less than 20% of cells), weakly positive (+1, strongly-positive staining present in more than 20% of cells, or weakly-positive staining in more than 50% of cells), and strongly-positive (+2, strongly-positive staining in more than 50% of cells). Scoring of IHC findings was based on previous studies from our group. For analysis, patients with tumors showing a weak (+1) or strong (+2) immunoreactivity were assigned to the MV-positive group. In order to increase the stringency of the MV assay, a case was considered positive for MV, if both anti-nucleoprotein and anti-hemagglutinin staining were positive.

Statistical analysis: the following parameters were converted into dichotomic variables for analysis of MV status: ethnicity, place of birth, age at diagnosis, immigration after 1988, tumor size, lymph node involvement, histological grade, DCIS component, pathological complete response after neo-adjuvant chemotherapy; ER, PR, and HER2/neu status, p53, and ki67. Variables were evaluated by the two-tailed χ2-test or two tailed Fisher's exact test. The results of the univariate analysis were used to determine the variables that were included in the subsequent multivariate analysis. Variables that were significantly associated with MV positive status were evaluated by multivariate logistic regression analysis. Using a stepwise selection method, a multivariate, logistic regression model was constructed to identify predictors for MV status with adjustment for relevant demographic, pathologic and immunohistochemical variables. All statistical tests were two-sided, and statistical significance was defined as P<0.05. Statistical analyses were performed using the STATA® software (STATA for Windows, version release 9.0, StataCorp LP., Texas, USA). Table 2 shows the assignment of each dichotomic variable constructed for analysis.

TABLE 2
Dichotomic assignment of demographic and clinicopathological
variables.

| | Variable Name | Dichotomic Variable Assignment | |
|---|---|---|---|
| Demographic | Age at diagnosis | ≦50 years | >50 years |
| variables | Ethnic origin | Jewish | Bedouin- |
| | | | Arab |
| | Place of birth | Israel/Europe/ | Asia/Africa |
| | | America | |
| Pathologic | Tumor diameter | <10 mm | >10 mm |
| variables | Axillary lymph node | No involvement | Involvement |
| | involvement | (pN0 or pNmic) | (pN1-3) |
| | pCR (pathological | Yes | No |
| | complete response | | |
| | after neoadjuvant | | |
| | chemotherapy) | | |
| | DCIS component | Yes | No |
| | Histological grade | Low or intermediate | High (G3) |
| | | (G1-2) | |
| Immuno- | Estrogen receptor | Positive | Negative |
| histochemical | (ER) status | | |
| stains | Progesterone | Positive | Negative |
| | receptor (PR) status | | |
| | HER2/neu status | Positive | Negative |
| | Ki67 status | Positive | Negative |
| | P53 status | Positive | Negative |
| | Measles virus | Positive | Negative |
| | hemagglutinin | | |
| | Measles virus | Positive | Negative |
| | nucleoprotein | | |
| | Positive staining for | Positive | Negative |
| | both measles | | |
| | proteins | | |

### Results

Demographics: 131 patients with a diagnosis of limited or locally-advanced breast cancer diagnosed at the Soroka Medical Center during the years 1998-2005 were included in the study. Complete demographic, clinical and pathologic data was available for all patients. 112 patients were diagnosed with limited disease and underwent primary surgical procedure. 19 patients were diagnosed with locally-advanced disease and received neo-adjuvant chemotherapy before surgical intervention. This group of patients was excluded from correlation analyses for tumor size or lymph node involvement but was evaluated for response to chemotherapy. The average age of the whole study population was 48.6 years (range 27 to 79 years). Hormone receptors, either to estrogen or to progesterone were detected in 72 patients (55%). The age distribution of patients with estrogen receptor positive breast cancer (49.4 years) was similar to patients with estrogen receptor negative breast cancer (47.8 years). 81 patients aged 50 years or younger and 50 patients aged above 50 years. 63 patients (48%) were born in European countries (or in America) and 41 patients (31%) were born in Israel. 41 of the 63 patients born in Europe/America immigrated to Israel after 1988 (“new comers”). Only 9 of the 131 patients (6.8%) were Bedouin-Arabs, The demographic and clinicopathological characteristics of the patients according to their estrogen-receptor status are shown in Table 3.

TABLE 3
Demographic and clinicopathological characteristics of patients
according to their estrogen-receptor status.

| | | COHORT (N = 131)* | |
|---|---|---|---|
| | | ER-positive | ER-negative |
| Characteristics | | 54% (71) | 46% (60) |
| Age at diagnosis | Average** | 49.4 ± 10.1 | 47.8 ± 8.6 |
| (years) | Median | 49 | 48 |
| | Range | 27-79 | 27-83 |
| Age distribution | ≦50 | 62% (44) | 62% (37) |
| (years) | >50 | 38% (27) | 38% (27) |
| | ≦35 | 6% (4) | 5% (3) |
| | >35 | 94% (67) | 95% (57) |
| Place of birth | Israel | 31.0% (22) | 31.7% (19) |
| | Europe/America | 47.9% (34) | 48.3% (29) |
| | Africa/Asia | 21.1% (15) | 18.3% (11) |
| | Unknown | — | 1.7% (1) |
| Ethnic origin | Jewish | 94% (67) | 95% (55) |
| | Bedouin-Arab | 6% (4) | 5% (5) |
| Immigration date | Before 1988 | 35% (12) | 38% (11) |
| (For | After 1988 (including) | 65% (22) | 62% (18) |
| Europe/America- | | | |
| born) | | | |
| Surgical staging | Primary | 86% (61) | 85% (50) |
| | After neo-adjuvant Rx | 14% (10) | 15% (9) |
| Sentinel lymph | Yes | 41% (29) | 54% (32) |
| node biopsy | No | 59% (42) | 46% (27) |
| Axillary lymph | Yes | 56% (40) | 61% (36) |
| node dissection | No | 44% (31) | 39% (23) |
| Tumor size | T<10 mm | 64.5% (40) | 50% (26) |
| (Primary staging | T>10 mm | 35.5% (22) | 50% (26) |
| only) | | | |
| Lymph node | pNmic | — | — |
| involvement | pN0 | 56.7% (34) | 53.8% (28) |
| (Primary staging | pN1 (1-3 nodes) | 30% (18) | 32.7% (17) |
| only) | pN2 (4-9 nodes) | 13.3% (8) | 11.5% (6) |
| | pN3 (>9 nodes) | — | 1.9% (1) |
| Pathologic | Yes | 0 | 11% (1) |
| complete response | No | 100% (10) | 89% (9) |
| (After neo- | | | |
| adjuvant Rx) | | | |
| DCIS component | Yes | 30% (21) | 53% (32) |
| | No | 70% (50) | 47% (28) |
| Pathologic grade | Low or intermediate | 70% (50) | 18% (11) |
| | (G1-2) | | |
| | High (G3) | 30% (21) | 82% (49) |
| PR status | Positive | 83% (59) | 1.7% (1) |
| | Negative | 17% (12) | 98.3% (59) |
| Ki67 status | Positive | 2.8% (2) | 37% (22) |
| | Negative | 97.2% (69) | 63% (38) |
| p53 status | Positive | 20% (14) | 48% (28) |
| | Negative | 80% (56) | 52% (31) |
| HER2/neu status | Positive | 10% (7) | 27% (16) |
| | Negative | 90% (64) | 73% (44) |
| Measles virus | Positive | 82% (58) | 70% (42) |
| hemagglutinin | Negative | 18% (13) | 30% (18) |
| Measles virus | Positive | 90% (64) | 72% (43) |
| nucleoprotein | Negative | 10% (7) | 28% (17) |
| Staining for both | Positive | 73% (52) | 53% (32) |
| measles virus | Negative | 27% (19) | 47% (28) |
| proteins | | | |

*Percentages may not total 100 because of rounding.
**Plus-minus values are means + SD.

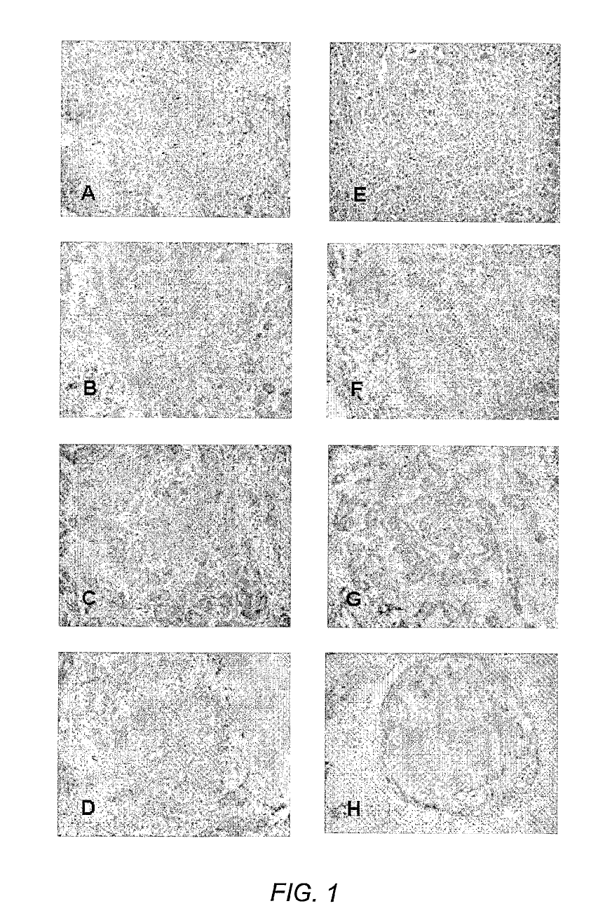

Clinicopathological results: 61 patients (46.5%) underwent sentinel lymph node biopsy during the study period. 76 patients (58%) underwent axillary lymph node dissection. In 48 of 114 patients, the tumor sized more than 10 mm. A DCIS component was found in 53 of 131 patients (40.5%). 62 of 112 patients (47%) showed regional lymph node involvement. 19 patients received primary, neo-adjuvant chemotherapy, of whom one patient showed complete pathological response. Low or intermediate pathologic grade (G1-2) was found in 50 of 71 patients (70.4%) with estrogen receptor positive tumor, and in 11 of 60 patients (18.3%) with estrogen receptor negative tumors. In 60 of 131 patients (46%) the progesterone receptor was positive. Only one patient had progesterone receptor-positive, estrogen receptor-negative tumor. Positive ki67 staining was found in 22 of 60 patients (37%) with estrogen receptor negative tumors compared with two of 71 patients (3%) with estrogen receptor positive tumors. Overexpression of p53 was found in 28 of 60 patients (47.5%) with estrogen receptor negative tumors, compared with 14 of 70 patients (20%) with estrogen receptor positive tumors. Positive HER2/neu status was found in 16 of 60 patients (27%) with estrogen receptor negative tumors, compared with 7 of 71 patients (10%) with estrogen receptor positive tumors. Positive staining for MV-hemagglutinin was found in 100 of 131 patients (76%), and for MV-nucleoprotein in 107 of 131 patients (82%). Positive staining for MV antigens, hemagglutinin and nucleoprotein was found in 84 patients (64%). Staining for both MV antigens was cytoplasmatic in all breast cancer biopsies. All biopsies containing DCIS component (40% of cases) showed MV in DCIS in addition to the presence of MV antigens in the invasive component. FIG. 1 shows degrees of positive immunohistochemical staining (+1, weakly-positive; +2, strongly-positive) for MV-hemagglutinin and MV-nucleoprotein in breast cancer biopsies.

Statistical analysis: In univariate analysis, positive MV status (positive staining for both MV antigens) correlated with estrogen receptor positive status (P=0.018), negative ki67 status (P=0.029), low to intermediate pathologic grade (G1-2) (P=0.037), young age (P=0.039), progesterone receptor positive status (P=0.043), and positive p53 status (P=0.049). No correlation was found between positive MV status and place of birth, ethnic origin, immigration period, tumor size, regional lymph node involvement, DCIS component in the biopsy, or positive HER2/neu status. Table 4 illustrates factors associated with positive MV status.

TABLE 4
Factors associated with measles virus positive status.

| | | Measles | Measles | |
|---|---|---|---|---|
| | | virus | virus | |
| Variable | | positive* | negative* | Pv |
| | | 64% (84) | 36% (47) | |
| Place of birth | Israel/Europe/ | 63% (66) | 37% (39) | 0.54 |
| | America | | | |
| | Africa/Asia | 69% (18) | 31% (8) | |
| Ethnic origin | Jewish | 64.5% (78) | 35.5% (43) | 0.89 |
| | Bedouin-Arab | 66% (6) | 33% (3) | |
| Age at diagnosis | ≦50 | 69% (56) | 31% (25) | 0.13 |
| (years) | >50 | 56% (28) | 44% (22) | |
| Tumor size | <10 mm (T1a, b) | 63% (41) | 37% (24) | 0.64 |
| | >10 mm | 67% (31) | 33% (15) | |
| Lymph node | Yes | 69% (44) | 31% (20) | 0.052 |
| involvement | No | 58% (28) | 42% (20) | |
| Pathologic grade | Low or | 75% (42) | 25% (14) | 0.037 |
| | intermediate | | | |
| | (G1-2) | | | |
| | High (G3) | 57% (40) | 43% (30) | |
| DCIS | Yes | 59% (31) | 41% (22) | 0.27 |
| component | No | 68% (53) | 32% (25) | |
| ER status | Positive | 73% (52) | 27% (19) | 0.018 |
| | Negative | 53% (32) | 47% (28) | |
| PR status | Positive | 73% (44) | 27% (16) | 0.043 |
| | Negative | 45% (40) | 44% (31) | |
| HER2/neu status | Positive | 57% (13) | 43% (10) | 0.37 |
| | Negative | 66% (69) | 34% (35) | |
| p53 status | Positive | 70% (61) | 30% (26) | 0.049 |
| | Negative | 52% (22) | 48% (20) | |
| Ki67 | >40% | 52% (25) | 48% (23) | 0.029 |
| positive stain | <40% | 71% (59) | 29% (24) | |
| (% of cells) | | | | |

Percentages may not total 100 because of rounding.

Stepwise multivariate regression analysis resulted in the delineation of the optimal set of variables that could be included in the equation: (1) age 50 years or younger (yes=1, no=0), (2) low or intermediate histological grade (yes=1, no=0), (3) ki67 index of less than 40% (yes=1, no=0), (4) positive ER status (yes=1, no=0), (5) positive PR status (yes=1, no=0), (6) positive p53 status (yes=1, no=0), (7) lymph node involvement (yes=1, no=0). The logistic regression model provided the estimated probability of positive measles status for a particular patient with breast cancer. The model estimated the following statistically significant contributors to the probability of positive measles status: age (50 years or younger), histological grade (low or intermediate), positive p53 status. In multivariate, logistic analysis the following variables showed association of statistical significance with positive MV-status: low and intermediate grade (P=0.012), positive p53 staining (P=0.033), and age of 50 years or younger (P=0.041). Table 5 shows the results of logistic regression analysis in patients with breast cancer.

TABLE 5
Factors associated with measles virus positive status
in multivariate, logistic regression analysis.

| Variable | Odds Ratio | P Value | 95% C.I. |
|---|---|---|---|
| Low and intermediate grade | 3.04 | 0.012 | 1.28-7.23 |
| P53 positive staining | 2.59 | 0.033 | 1.08-6.18 |
| Age, 50 years or younger | 2.44 | 0.041 | 1.04-5.73 |

### CONCLUSION

A study which included a relatively large number of breast cancer patients found measles virus antigens in over half the patients. In multivariate logistic analysis, MV status was associated with low to intermediate histological grade of tumors, p53 positive status, and age of 50 years or younger. Since an age of 50 years or younger is regarded as a good surrogate for menopausal status, MV positive status is probably associated with premenopausal status. In conclusion, MV was detected in a large proportion of breast cancer biopsies. Its finding is associated with younger age of patients, and with important biologic characteristics such as lower histological grade and p53 positive staining.

### Example 2

### Evidence for Association of Measles Virus with Lung Cancer

A cross-sectional, retrospective, clinicopathological study on patients with breast cancer was performed in order to find evidence for a possible association of MV with breast cancer.

Patients and Methods

A cross-sectional, retrospective, clinicopathological study was performed on patients with non small-cell lung cancer (NSCLC) with aimed at determining the presence of measles antigens in tumors and the prognostic importance of measles and Pirh2 in these patients.

Study population: the study population included newly-diagnosed patients with NSCLC of all stages during the years 1996-2006, for whom adequate pathological specimens and clinical data meeting study requirements were available. Cases were selected based on availability of tissue and were not stratified for known preoperative or pathological prognostic factors. The median follow-up time was 38 months (range 3-127 months). Exclusion criteria were patients with histological diagnosis of lung cancer other than NSCLC, a previous history of another primary tumor, or patients who had previously been treated with neo-adjuvant chemotherapy and/or radiotherapy. The list of patients was obtained from the computerized file of all patients with NSCLC referred to the department of oncology during the study period. For each patient, data were extracted from the case file in the department of oncology, and the hospital computerized file. Data encompassed the following parameters: 1) demographic data including age at diagnosis, gender, ethnicity, and place of birth. 2) clinical characteristics including a history of tobacco smoking, extent of anatomic involvement, and survival; the survival time was calculated from date of diagnosis to date of death. 3) pathological information including histological identification as recommended by the World Health Organization, tumor size, local invasion, and lymph node metastasis. Tumor size was evaluated in freshly obtained tissue, before formalin fixation, and coded according to the International Union Against Cancer pT recommendations. The final disease stage was determined by a combination of clinical, surgical and pathologic findings, according to the current tumor-node-metastasis staging system for NSCLC. Whenever adequate surgery was performed, pathological staging dominated. All cases were reevaluated for histological type by two pathologists. For each case, a representative paraffin block containing tumor was chosen and sections were taken for immunohistochemical (IHC) studies for MV antigens, hemagglutinin and nucleoprotein, p53, and Pirh2. Data were collected into EPIINFO software, 2002 version for Windows.

Pathology: paraffin-embedded, formalin-fixed surgical specimens were collected, and serial sections (4 μm) containing representative malignant cells were stained with hematoxylin and eosin and classified based on the World Health Organization criteria.

The presence of tumor cells, as well as the cellularity of the tumor was evaluated, and a biopsy sample was considered eligible for the study only if tumor morphology was well preserved. A total of 65 tumor samples were classified as NSCLC counting 34 cases of adenocarcinoma (including two cases of bronchoalveolar carcinoma), 21 cases of squamous cell carcinoma, and 10 cases of large cell carcinoma (including 4 cases of large cell carcinoma with neuroendocrine features). Cases of small cell carcinoma, carcinoid or mixed small and non-small cell type were not included in the study. IHC studies for MV were performed using the avidin-biotin peroxidase complex (Envision, DAKO). Studies for p53 and Pirh2 were performed according to instructions from the manufacturers. Immunostaining was expressed as the percentage of labeled nuclear area over the total neoplastic nuclear area in the section.

Immunochemistry: Immunohistochemical staining (IHC) by the avidin-biotin peroxidase complex method was performed using the same procedure as described in example 1.

Antibodies: antibodies against MV nucleoprotein, MV hemagglutinin, and p53 were the same as described in example 1 and used according to the same procedure. For expression and localization of Pirh2 we used a rabbit polyclonal anti-Pirh2 at a dilution of 1:100 (clone BL588; Bethyl, USA). Pirh2 was observed primarily in the cytoplasm and plasma membrane but rarely also in the nucleus. Expression was considered positive if more than 10% of cells stained strongly-positively for Pirh2.

Scoring system for MV antigens. The cytoplasmic intensity of MV hemagglutinin and MV nucleoprotein were evaluated separately and scored semi-quantitatively as negative (0, no expression or weakly-positive staining in less than or equal to 30% of the cells, or strongly-positive in less than 20% of cells), weakly positive (+1, strongly-positive staining present in more than 20% of cells, or weakly-positive staining in more than 50% of cells), and strongly-positive (+2, strongly-positive staining in more than 50% of cells). Scoring IHC findings was based on previous studies from our group. For analysis, patients with tumors showing a weak (+1) or strong (+2) immunoreactivity were assigned to the MV-positive group. In order to increase the stringency of the MV assay, a case was considered positive for MV, if both anti-nucleoprotein and anti-hemagglutinin staining were positive. IHC assays were scored jointly by two pathologists.

Statistical analysis: the following parameters were converted into dichotomic variables for analysis of MV status: gender, ethnicity, place of birth, age at diagnosis, smoking status, histological type, tumor size, lymph node involvement, stage, Pirh2 and p53. Variables were evaluated by the two-tailed χ2-test or two tailed Fisher's exact test. The results of the univariate analysis were used to determine the parameters that were included in the subsequent multivariate, logistic regression analysis.

Survival time was measured from the date of surgical diagnosis to the date of death or last follow-up examination. Patients who were alive or have died of a cause other than lung cancer were censored for analysis of disease specific survival, whereas patients who were alive or had died of any cause other than lung cancer were censored for analysis of overall survival. Survival curves were estimated by Kaplan and Meier's method. Overall survival analyses were performed by applying univariate and stepwise forward multivariate Cox proportional hazards regression models. All statistical tests were two-sided, and statistical significance was defined as P<0.05. Statistical analyses were performed using the STATA® software (STATA for Windows, version release 9.0, StataCorp LP., Texas, USA). Table 6 shows the dichotomic assignment of each variable constructed for analysis.

TABLE 6
dichotomic assignment of demographic, clinicopathologic, and
immunohistochemical variables in patients with non-small cell
lung cancer.

| | Variable | Dichotomic Variable Assignment | |
|---|---|---|---|
| Demographic | Age at diagnosis | ≦60 years | >60 years |
| variables | Gender | male | female |
| | Ethnic origin | Jewish | Bedouin-Arab |
| | Place of birth | Israel/Europe/ | Asia/Africa |
| | | America | |
| Clinico- | Smoking status | Yes | No |
| pathologic | Tumor diameter | T1/T2 | T3/T4 |
| variables | Lymph node | Yes | No |
| | metastasis | | |
| | Stage | Limited, 1 or 2 | Advanced, |
| | | | 3 or 4 |
| | Histological type | Adenocarcinoma | Other types |
| Immuno- | MV-hemagglutinin | Positive | Negative |
| histochemical | MV-nucleoprotein | Positive | Negative |
| variables | P53 status | Positive | Negative |
| | Pirh2 status | Positive | Negative |
| | MV-status | Positive | Negative |

### Results

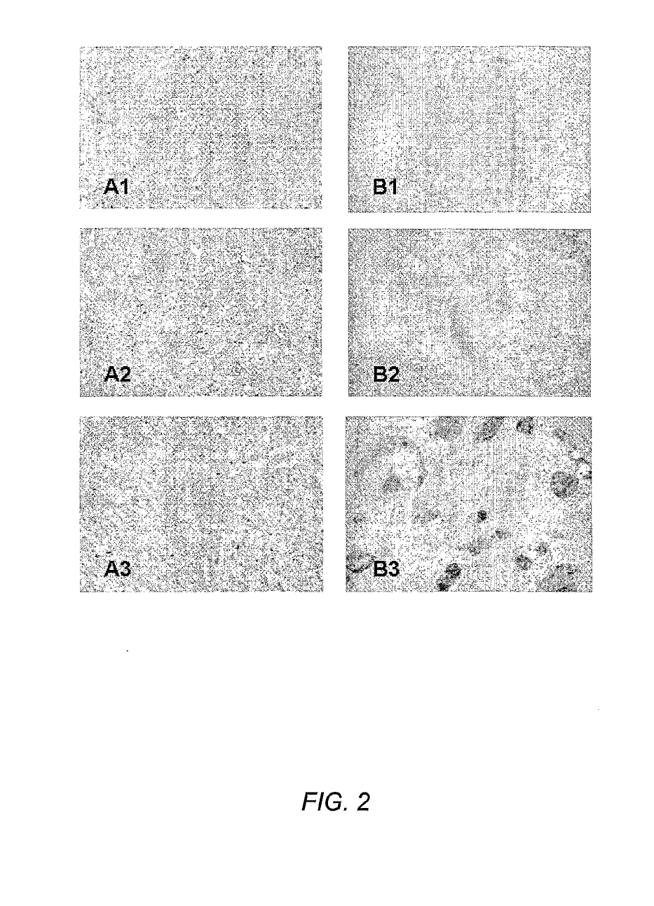

Complete demographic, clinicopathological, and immunohistochemical data are presented in table 7. Sixty five patients with a histological diagnosis of NSCLC were included in the study. The median age was 65 years (range 40-84 years). 47 patients (72%) were males and 18 patients (28%) were females. 61 patients (94%) were Jewish and only 4 patients (6%) were Bedouin-Arabs. Most Jewish patients were born in Israel/Europe/America (44 of 61, 72%) and were of Ashkenazi extraction. 17 of 61 (28%) of the Jewish patients were born in Asia/Africa and were of Sephardic extraction. Almost all patients (90%) were smokers. Most patients had early-lung cancer; 34 patients (54%) had stage 1 disease, and only 5 patients (8%) were diagnosed in stage 4 disease. 34 patients (52%) were diagnosed with adenocarcinoma, 21 patients (32%) had squamous cell carcinoma, and only 10 patients (15%) had large cell carcinoma with, or without neuroendocrine features. Most patients underwent lobectomy (49 patients, 75%) and only 4 patients (6%) had a biopsy only. Lymph nodes were involved in 33 patients (47%). 32 patients (49%) developed distant metastasis in addition to the five patients who were diagnosed in stage 4 disease. Measles IHC for nucleoprotein was positive in 48 patients (74%), for hemagglutinin positive in 41 patients (64%), and for either nucleoprotein or hemagglutinin in 54 patients (83%). Measles status (expression of both MV antigens) was positive in 35 patients (54%). p53 expression was positive in 32 patients (40%), and Pirh2 expression was positive in 33 patients (53%). Staining for both MV antigens was cytoplasmic in all LC biopsies with a similar pattern of staining in all three histological types. Staining for MV-hemagglutinin showed more background staining than for MV-nucleoprotein. FIG. 2 shows degrees of positive immunohistochemical staining (+1-weakly-positive; +2-strongly-positive) for MV-hemagglutinin and MV-nucleoprotein in LC of the three histological types: adenocarcinoma, squamous cell carcinoma, and large cell carcinoma.

TABLE 7
demographic, clinicopathological and immunohistochemical
characteristics of patients with non-small cell lung cancer.

| Parameter | | Cohort (N = 65) |
|---|---|---|
| Age - years | Median | 65 |
| | Range | 40-84 |
| | Mean | 65 + 9.5 |
| Gender | Male | 72% (47) |
| | Female | 28% (18) |
| | Male to female ratio | 2.61 |
| Ethnicity | Jewish | 54% (61) |
| | Bedouin-Arab | 6% (4) |
| Place of birth (for Jewish | Israel | 11% (7/61) |
| ethnicity) | Europe/America | 61% (37/61) |
| | Asia/Africa | 28% (17/61) |
| Tobacco history | Yes | 90% (59) |
| | No | 9% (6) |
| Histology | Adenocarcinoma | 52% (34) |
| | Squamous | 32% (21) |
| | Large-cell | 15% (10) |
| Primary stage | 1 | 54% (35) |
| | 2 | 29% (19) |
| | 3 | 9% (6) |
| | 4 | 8% (5) |
| Primary surgery | Segmentectomy | 8% (5) |
| | Lobectomy | 75% (49) |
| | Pneumonectomy | 11% (7) |
| | Biopsy only | 6% (4) |
| Chemotherapy (metastatic | Yes | 43% (16/37) |
| disease) | No | 57% (21/37) |
| Late recurrence | Yes | 53% (32/60) |
| | No | 47% (28/60) |
| Nodal status | Positive | 53% (33/62) |
| | Negative | 47% (29/62) |
| MV - hemagglutinin | Positive | 63% (41/65) |
| | Negative | 37% (24/65) |
| MV - nucleoprotein | Positive | 74% (48/65) |
| | Negative | 26% (17/65) |
| MV - status | Positive | 54% (35/65) |
| | Negative | 46% (30/65) |
| p53 status | Positive | 51% (32/62) |
| | Negative | 49% (31/62) |
| Pirh2 status | Positive | 53% (33/62) |
| | Negative | 47% (29/62) |

Statistical analysis: in univariate analysis (table 8), positive MV status (positive expression of both MV antigens) correlated with age above 60 years (p=0.018), and expression of Pirh2 (p=0.006), and for Jewish patients equivocally with Israel/Europe/America place of birth (p=0.051). MV status was not associated with gender, ethnicity, smoking status, tumor extent, nodal status, stage, histological type, or p53 status. Pirh2 expression correlated with positive nodal status (p=0.024), but not with age, tumor size, stage, histological type or p53 status. Table 8 illustrates factors associated with positive MV status.

TABLE 8
factors associated with measles virus positive status in patients
with non-small cell lung cancer.

| | | MEASLES | MEASLES | |
|---|---|---|---|---|
| | | VIRUS | VIRUS | |
| | | POSITIVE | NEGATIVE | |
| | | 54% (35) | 46% (30) | |
| VARIABLE | | Total n = 65 | | P |
| Gender | Female | 45% (8) | 55% (10) | 0.35 |
| | Male | 57% (27) | 43% (20) | |
| Age | ≦60 years | 30% (5) | 70% (12) | 0.018 |
| | >60 years | 63% (30) | 37% (18) | |
| Ethnicity | Jewish | 56% (34) | 44% (27) | 0.26 |
| | Bedouin-Arab | 25% (1) | 75% (3) | |
| Place of birth | Israel/Europe/ | 48% (21) | 52% (23) | 0.051 |
| (Jewish only) | America | | | |
| | Africa/Asia | 76% (13) | 24% (4) | |
| Smoking status | Yes | 53% (26) | 47% (23) | 0.48 |
| | No | 60% (6) | 40% (4) | |
| Tumor extent | T1 or T2 | 59% (24) | 41% (17) | 0.75 |
| | T3 or T4 | 50% (11) | 50% (11) | |
| Nodal status | negative | 59% (24) | 41% (17) | 0.52 |
| | positive | 50% (11) | 50% (11) | |
| Stage | Stage 1 or 2 | 57% (31) | 43% (23) | 0.20 |
| | Stage 3 or 4 | 36% (4) | 64% (7) | |
| Tumor type | Adenocarcinoma | 55% (18) | 45% (15) | 0.91 |
| | Other types | 53% (17) | 47% (15) | |
| p53 status | Positive | 62% (20) | 38% (12) | 0.26 |
| | Negative | 48% (15) | 50% (16) | |
| Pirh2 | Positive | 70% (23) | 30% (10) | 0.006 |
| | Negative | 34% (10) | 66% (19) | |

Stepwise multivariate regression analysis resulted in the delineation of the optimal set of variables that could be included in the equation: (1) age of 60 years or older (yes=1, no=0), (2) Africa/Asia place of birth (yes=1, no=0), (3) adenocarcinoma (yes=1, no=0), (4) early stage (yes=1, no=0), (5) p53 positive status (yes=1, no=0), (6) Pirh2 positive status (yes=1, no=0), (7) lymph node involvement (yes=1, no=0). The logistic regression model provided the estimated probability of positive measles status for a particular patient with NSCLC. In multivariate logistic analysis the following variables showed association of statistical significance with positive MV status: age of 60 years or older (P=0.01) and Pirh2 positive status (P=0.014). Table 9 shows factors associated with measles virus positive status in patients with NSCLC in multivariate logistic regression analysis.

TABLE 9
multivariate logistic regression analysis of factors associated
with measles virus positive status in all patients with non-
small cell lung cancer.

| Variable | Odds-Ratio | P Value | 95% C.I. |
|---|---|---|---|
| Age (60 yrs or older) | 1.09 | 0.010 | 1.02-1.17 |
| Pirh2 positive status | 6.12 | 0.014 | 1.45-25.92 |
| p53 positive status | 3.27 | 0.095* | 0.81-13.12 |

*Not significant statistically

In Cox multivariate analysis, the only variables associated with improved survival were Pirh2 positive status (P=0.027), Africa/Asia place of birth (P=0.031), and early stage (P=0.036).

### CONCLUSION

A study which included a number of non small cell lung cancer patients found measles virus antigens in over half the patients. In this study, expression of either one of the MV antigens was detected in 83% of biopsies, and expression of both antigens defined as positive MV status was determined in 53% of patients. Such a high rate of detection may suggest a role played by MV in the development of NSCLC. MV status was not associated with tumor size or with lymph node involvement, both important prognostic factors in lung cancer. However, positive MV status was associated with age of patients with higher expression in patients older than 60 years. Measles is ubiquitous and most of the adult population has been exposed to either wild-type MV or to attenuated virus used in vaccination. All patients belonging to the older age group were most likely exposed to the wild type MV, and were not vaccinated with an attenuated MV. In conclusion, this study provides evidence of the presence of MV antigens in a large proportion of NSCLC biopsies and suggests a possible association between MV and NSCLC. MV status was linked to older age of patients, and to expression of Pirh2.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

What is claimed is:

1. A method for treating cancer in a patient in need thereof, the method comprising:
a) testing cancer cells of the patient using a measles virus immunoassay,
b) detecting the presence of measles virus-immunoreactive cancer cells,
c) if the patient is determined to have measles virus-immunoreactive cancer cells, administering an immunogenic composition that elicits an immune response to measles virus to the patient.

2. The method of claim 1, wherein the cancer cells are present in a sample obtained from the patient.

3. The method of claim 2, wherein the measles virus immunoreactivity of the cancer cells in said sample is determined by immunohistochemistry.

4. The method of claim 3, wherein the immunohistochemical antigen marker is measles virus nucleoprotein (NP) or measles virus hemagglutinin (HA).

5. The method of claim 1, wherein the immunogenic composition comprises at least one of the group consisting of: RNA-free virus-like particles, at least one isolated measles virus protein or portion thereof, a measles virus DNA vaccine, a killed measles virus and an attenuated measles virus.

6. The method of claim 5, wherein the isolated measles virus protein is selected from the group consisting of: the nucleoprotein (NP), the polymerase cofactor phosphoprotein (P), the matrix (M), the fusion (F), the hemagglutinin (HA), and the large RNA polymerase (L) proteins.

7. The method of claim 5, wherein the killed measles virus or the attenuated measles virus is selected from the group consisting of the Edmonston Zagreb measles strain, the Edmonston-Enders strain, the Moraten strain, and the Moraten Berna strain.

8. The method of claim 1, wherein the immunogenic composition is provided within a commercial vaccine formulation.

9. The method of claim 1, wherein the immunogenic composition comprises an edible vaccine.

10. The method of claim 1, wherein the immunogenic composition further comprises at least one of: an adjuvant and a toll-like receptor inducer.

11. The method of claim 1, wherein the immunogenic composition is administered intramuscularly, subcutaneously, transdermally, orally, or intranasally.

12. The method of claim 1, wherein the cancer is selected from the group consisting of: brain cancer, prostate cancer, breast cancer, skin cancer, colon cancer, rectal cancer, lung cancer, pancreatic cancer, Hodgkin's lymphoma, non-Hodgkin lymphoma, multiple myeloma, leukemia, oesophageal cancer, renal cancer, uterine, endometrial and cervical cancer, ovarian cancer, testicular cancer, urinary bladder cancer, gastric cancer, liver cancer, thyroid cancer, and head and neck cancer.

13. The method of claim 1, wherein the cancer is breast cancer or non small-cell lung cancer.

14. The method of claim 1, further comprising administering at least one anti-cancer therapy.

15. The method of claim 14, wherein the anti-cancer therapy is selected from the group consisting of surgery, chemotherapy, radiotherapy, hormone therapy, therapy with immunomodulatory agents, and therapy with targeted or antiangiogenic agents.

16. A method for treating cancer in a patient in need thereof, the method comprising:
a) obtaining a sample of cancer cells from said patient,
b) testing said sample for the presence of measles virus-specific mRNA,
c) detecting the presence of measles virus-positive cancer cells,
d) if the patient is determined to have measles virus-positive cancer cells, administering an immunogenic composition that elicits an immune response to measles virus to the patient.

17. The method of claim 16, wherein the measles virus positive cancer cells in said sample are detected by reverse transcriptase-PCR or by in situ hybridization.
